(19) United States
(12) Patent Application Publication
(10) Pub. No.: US 2026/0179787 A1
(43) Pub. Date: Jun. 25, 2026

(54) PROVISION OF IDENTIFICATION DATA FOR A SECOND ENTITY

(71) Applicant: Siemens Healthineers AG, Forchheim (DE)

(72) Inventors: Alois Regensburger, Poxdorf (DE); Philipp Roser, Allston, MA (US)

(21) Appl. No.: 19/422,785

(22) Filed: Dec. 17, 2025

(30) Foreign Application Priority Data
Dec. 19, 2024 (DE) 10 2024 212 182.7

(51) Int. Cl. G16H 80/00

(52) U.S. Cl. CPC G16H 80/00

(57) ABSTRACT

A computer-implemented method for providing identification data for a second entity includes: capturing examination data from an imaging-based examination of an examination object by a first entity; providing an abstract vector by applying a trained function to input data, which is based on the examination data of the first entity, wherein the trained function is based on an abstract vector model; and providing the identification data for the second entity on the basis of a similarity comparison of the abstract vector with other abstract vectors in a common abstract vector space stored in a collection, wherein respective identification data is assigned to the stored other abstract vectors. The present disclosure further relates to a computer-implemented method for providing a collection of abstract vectors, a computer-implemented method for providing a trained function, a provision unit, a system, and a computer program product.

The present patent document claims the benefit of German Patent Application No. 10 2024 212 182.7, filed Dec. 19, 2024, which is hereby incorporated by reference in its entirety.

### TECHNICAL FIELD

The present disclosure relates to a computer-implemented method for providing identification data for a second entity, a computer-implemented method for providing a collection of abstract vectors, a computer-implemented method for providing a trained function, a provision unit, a system, and a computer program product.

### BACKGROUND

In medical imaging, examination objects such as patients may be examined using imaging-based procedures. These examinations deliver valuable information for diagnosis and treatment. This results in large amounts of examination data, which may also include findings, anamneses, and/or technical parameters in addition to the actual image data. In order to provide the best possible care for the examination objects, it may be useful to get a second opinion from a specialist in complex or rare cases.

Identifying a suitable specialist for a second opinion constitutes a challenge, however. The choice may be based on personal contacts or general specialty information, which may not lead to optimal expertise for the specific case. Additionally, it may be a laborious task to compile all the relevant examination data manually, in order to find a specialist. This may lead to delays in the treatment process.

Another problem is that the expertise of specialists may only be available locally and may not be used efficiently beyond institutional boundaries. This leads to suboptimal use of existing specialist knowledge and may compromise treatment quality, particularly in regions with limited access to specialists.

### SUMMARY AND DESCRIPTION

It is thus the object of the present disclosure to facilitate improved identification of a second entity for imaging-based examinations.

The scope of the present disclosure is defined solely by the appended claims and is not affected to any degree by the statements within this summary. The present embodiments may obviate one or more of the drawbacks or limitations in the related art.

The solution is described below in reference to the claimed method and in reference to the claimed systems. Features, advantages, or alternative embodiments may be assigned to each of the other claimed subject matters. In other words, claims and embodiments for the systems may be improved by features that are described or claimed in connection with the respective method. In this instance, the functional features of the method are implemented by physical units of the system.

Moreover, the solution is described below in relation to methods and systems for providing identification data for a second entity and/or methods and systems for providing a collection of abstract vectors, as well as in relation to methods and systems for providing a trained function. Features, advantages, or alternative embodiments may be assigned to each of the other claimed subject matters. In other words, claims and embodiments for providing a trained function may be improved by features that are described or claimed in connection to the provision of identification data for a second entity and/or the provision of a collection of abstract vectors. In particular, datasets that are used in the methods and systems for providing identification data for a second entity and/or methods and systems for providing a collection of abstract vectors may have the same properties and features as the corresponding datasets that are used in the methods and systems for providing a trained function, and the trained functions that are provided by the respective methods and systems may be used in the methods and systems for providing identification data for a second entity and/or methods and systems for providing a collection of abstract vectors.

The disclosure relates in a first aspect to a computer-implemented method for providing identification data for a second entity. In one act, examination data from an imaging-based examination of an examination object is captured by a first entity. In another act, an abstract vector is provided by applying a trained function to input data, which is based on the examination data of the first entity, wherein the trained function is based on an abstract vector model. In another act, the identification data for the second entity is provided on the basis of a similarity comparison of the abstract vector with other abstract vectors in a common abstract vector space stored in a collection, wherein respective identification data is assigned to the stored other abstract vectors.

Capturing the examination data may include recording and/or receiving the examination data. The examination data may be captured by a sensor and/or a medical imaging device. The examination data may be received in particular by capturing and/or reading from a computer-readable data memory and/or receiving from a data memory unit, for example, a database, particularly an image database. Furthermore, the examination data may be provided by a provision unit of one or more medical imaging devices, particularly using the same or different imaging modalities. The imaging-based examinations may include different medical imaging procedures, such as X-rays, computed tomography (CT), magnetic resonance imaging (MRI), ultrasound, positron emission tomography (PET), endoscopy and/or examinations using an external optical camera. The examination object may be a human and/or animal patient and/or an examination phantom, for example, a vascular phantom. Moreover, the examination data may be manually annotated, for example, by the first entity.

A first entity may be a medical entity, a medical facility, a competence center, a doctor, a medical specialist, and/or a technical specialist, which performs the imaging-based examination. The examination data may include medical image data, medical diagnostic data, medical anamnesis data, recording parameters of a medical imaging device, operating parameters of a medical object during the imaging-based examination, measured values of a medical monitoring device, a language of the medical diagnostic data and/or of the anamnesis data, metadata of the medical image data, and/or of the medical diagnostic data, and/or of the anamnesis data, and/or location information for the first entity.

An abstract vector, in particular an embedding vector, may constitute a mathematical representation of the examination data in a multi-dimensional space, for example, a multimodal embedding. The trained function may include a machine-learning model, which has been trained to map examination data to an abstract vector. The input data for the trained function is based on the examination data. In particular, the input data for the trained function may include the examination data. Advantageously, the trained function may have been provided by a proposed method for providing a trained function, which is described below. The abstract vector model may be a mathematical structure that describes the relationship between the input data, particularly the examination data, and the abstract vector.

The second entity may be a medical entity, a medical facility, a competence center, a doctor, a medical specialist, and/or a technical specialist, which is considered as a potential expert for a second opinion. The identification data may include contact information, specialties, level of experience, and/or other relevant information about the second entity.

The similarity comparison may be based on a comparison of a geometric distance measurement between the respective abstract vectors in the common abstract space. Alternatively, or additionally, the similarity comparison may be based on a cross-correlation, an overlap integral, and/or a count of matching entries between the respective abstract vectors in the common abstract vector space. Alternatively, or additionally, the similarity comparison may be based on artificial intelligence, for example, on a neural network and/or a large language model (LLM).

The collection of stored other abstract vectors with the respectively assigned identification data may be saved in a data memory unit, a database, and/or a cloud. A hierarchical comparison may thereby be performed, in which the abstract vectors are first compared on a higher level and then analyzed in more detail on a step-by-step basis. This may improve the efficiency of the similarity comparison.

Providing the identification data may include storage on a computer-readable storage medium, and/or displaying a graphic representation of the identification data on a display unit, and/or transferring to a provision unit, for example, in the form of a list and/or a profile.

The proposed computer-implemented method may facilitate accurate identification of experts for a second opinion, as the similarity of the respective examination data is compared in an abstract vector space. Accordingly, experts with experience of similar cases may be found, even if the descriptions of the cases may be different in natural language. The method may also improve the efficiency of the process of getting a second opinion, by automatically identifying relevant experts and providing their contact details.

In another advantageous embodiment of the computer-implemented method, the examination data may include at least one of the following elements: medical image data of the examination object, medical diagnostic data of the examination object, medical anamnesis data of the examination object, recording parameters of a medical imaging device for recording medical image data of the examination object, operating parameters of a medical object during the imaging-based examination of the examination object, measured values of a medical monitoring device, a language of the medical diagnostic data and/or of the anamnesis data, metadata of the medical image data, and/or of the medical diagnostic data, and/or of the anamnesis data, and/or location information for the first entity.

The medical image data of the examination object may include X-rays, particularly an individual X-ray image, and/or a series of X-rays, and/or a 3D volume image, computed tomography recordings, magnetic resonance recordings, ultrasound recordings, and/or recordings of the examination object taken by other imaging methods. This image data may include two-dimensional or three-dimensional representations of the examination object. The image data may also include time-resolved images of the examination object.

Medical diagnostic data of the examination object may include written or structured information about a health condition, diagnoses, recommended treatments, complications, treated pathologies, and/or other relevant medical findings, which were captured and/or received by the first entity.

Medical anamnesis data of the examination object may include information about medical history, earlier treatments, allergies, medication intake, or other relevant health information, which was provided by the examination object and/or captured and/or received by the first entity.

Recording parameters of a medical imaging device for recording medical image data of the examination object may include technical settings, such as exposure time, radiation dose, contrast medium administration, section thickness, and/or other device-specific parameters that are relevant for the image recording.

Operating parameters of a medical object during the imaging-based examination of the examination object may include information about a use and/or setting of medical instruments and/or devices during the examination, such as, for example, a positioning of a catheter, settings for an endoscope, and/or a configuration of a surgical robot. Additionally, the operating parameters may also include information for identifying the medical object that has been used, such as, for example, a kind, and/or type, and/or geometry, and/or material parameter, and/or origin, particularly a manufacturer, of the medical object. The medical object may include a surgical and/or diagnostic instrument, an implant, and/or a medical imaging device.

The recording parameters of the medical imaging device and/or the operating parameters of the medical object may be considered as device usage data in particular.

The medical monitoring device may advantageously be designed to monitor a condition of the examination object during the imaging-based examination. The measured values of the medical monitoring device may include a multitude of vital parameters, which enable a condition of the examination object to be monitored during the imaging-based examination. The measured values of the monitoring device may include an ECG signal (electrocardiogram signal), an EEG signal (electroencephalogram signal), a respiration monitor signal, particularly having information on breathing rate, breathing depth, and/or oxygen saturation, blood pressure information, body temperature information, and/or heart rate information of the examination object. The monitoring device may include one or more sensors, which are designed to capture the measured values of the examination object during the imaging-based examination, for example, ECG electrodes for measuring the electrical activity of the heart, EEG electrodes for monitoring brain activity, pulse oximeters for measuring oxygen saturation in the blood, blood pressure cuffs for measuring blood pressure non-invasively, thermistors for accurately recording body temperature, and/or chest straps or other heart rate sensors for continually monitoring heart rate.

The language of the medical diagnostic data and/or of the anamnesis data of the examination object may relate to the natural language in which this data is written, such as, for example, German, English, or another language. This information may be relevant for processing and interpreting the data.

Metadata of the medical image data, and/or of the medical diagnostic data, and/or of the anamnesis data may include additional structured information about the data itself, such as date of creation, author, clinical discipline, type of procedure, version number, and/or classifications according to medical coding systems.

Location information for the first entity may include geographic data and/or institutional assignments, which indicate where the examination was performed, such as, for example, the specific hospital, the medical facility, and/or the country.

Incorporating these varied data types in the examination data may create a comprehensive and detailed foundation for the application of the trained function. This may lead to a more precise provision of the abstract vector, particularly a multimodal embedding, which in turn may improve the accuracy of the similarity comparison and thus the relevance of the provided identification data for the second entity. Moreover, the variety of the data may enable complex medical cases to be captured in their entirety, thereby increasing the probability of identifying a second entity with relevant expertise for the specific case, particularly an existing case mix.

The examination data may advantageously also include a keyword search list. This keyword search list may be generated automatically on the basis of the other, particularly the remaining, types of examination data. For example, a keyword search list may be created automatically from identified image content on the imaging device or in an image database of the first entity. This keyword search list may serve as a profile for the first entity. Additionally, findings reports may be aggregated and automatically given keywords, in order to expand the keyword search list. Moreover, the first entity may have the opportunity to access the keyword search list manually. The first entity may supplement or correct the keyword search list, in order to further refine the search for a suitable second entity. This manual processing may enable highlighting of specific aspects of the case, which may not have been captured automatically, and/or removal of irrelevant keywords.

The use and potential manual adjustment of keyword search lists may increase the flexibility and accuracy of the method for identifying relevant second entities. It may create a bridge between automated analysis and human expertise, which may lead to an improved choice of experts for second opinions.

In another advantageous embodiment of the computer-implemented method for providing identification data for a second entity, some of the examination data coded in the abstract vector may be excluded from the similarity comparison.

The abstract vector may code different types of information that are derived from the examination data. This information may include medical image data, diagnostic data, anamnesis data, recording parameters, operating parameters, measured values of a medical monitoring device, language information, metadata, and/or location information. Some of this information may not be relevant or desired for the similarity comparison.

Excluding certain pieces of coded information from the similarity comparison may facilitate a more targeted and effective search for similar cases. For example, information that does not directly relate to a medical diagnosis or treatment, such as location data and/or specific recording parameters, may be excluded. This may help to focus the similarity comparison on the medically relevant aspects.

This information may be excluded in various ways. It is possible to exclude certain dimensions or components of the abstract vector when calculating the degree of similarity. Alternatively, the information to be excluded may already be left out when the trained function provides the abstract vector. In some cases, the various dimensions and/or components of the abstract vector may be weighted to adjust their relative significance for the similarity comparison.

The choice of the information to be excluded may be optimized on the basis of specialist medical knowledge, statistical analyses, and/or machine learning. This allows consideration to be given as to which information has the greatest influence on identifying relevant second entities.

Deliberately excluding certain pieces of information from the similarity comparison may improve the accuracy and relevance of the identified second entities. This may lead to a more effective use of the expert knowledge and improved support during medical decision-making.

In another advantageous embodiment of the computer-implemented method for providing identification data for a second entity, the other abstract vectors with the respectively assigned identification data may be stored in a data memory unit, a database, and/or a cloud.

Storing the other abstract vectors with the respectively assigned identification data in a data memory unit may facilitate efficient and fast storage and retrieval of the other abstract vectors with the respectively assigned identification data. A data memory unit may include hard disk space, a solid-state drive, and/or another non-volatile memory. Using a dedicated data memory unit may provide faster access to the stored other abstract vectors with the respectively assigned identification data, which may improve the speed of the similarity comparison.

Storing the other abstract vectors with the respectively assigned identification data in a database may facilitate structured and organized storage of the other abstract vectors with the respectively assigned identification data. A database may include a relational database, a NoSQL database, and/or a graph database. Using a database may offer advantages when managing large numbers of abstract vectors with the respectively assigned identification data, particularly if complex queries or indexing are required.

Storing the other abstract vectors with the respectively assigned identification data in a cloud may facilitate a flexible and scalable solution for storage and access to the other abstract vectors with the respectively assigned identification data. A cloud solution may include a cloud storage service or a cloud database. The advantage of using a cloud is that storage capacity may be expanded as needed and the other abstract vectors with the respectively assigned identification data may be accessed from different locations.

Furthermore, the other abstract vectors with the respectively assigned identification data for the reference units may be stored locally in a respective data structure for different units, particularly the reference entities and/or groups of multiple reference entities, for example, institutes. A central unit, particularly a server, may thereby act as a coordinator for the similarity comparison of the abstract vector with the other abstract vectors in the local data structures, particularly for sending the abstract vector for the similarity comparison to the local data structures and providing the identification data for the identified second entities.

The choice of specific storage solution for the other abstract vectors with the respectively assigned identification data may depend on various factors, such as, for example, the amount of data to be stored, the access speed requirements, the necessary scalability, and/or the security requirements. Using a combination of different storage solutions may be advantageous in some cases, in order to exploit the advantages of individual solutions.

Storing the other abstract vectors with the respectively assigned identification data in one of the named storage solutions may help to facilitate an efficient similarity comparison between the abstract vector of the first entity and the stored other abstract vectors. This may improve speed and accuracy when providing identification data for the second entity.

By using suitable storage solutions for the other abstract vectors, the computer-implemented method may achieve improved scalability, flexibility, and performance when providing identification data for a second entity.

In another advantageous embodiment of the computer-implemented method for providing identification data for a second entity, the similarity comparison may be based on a comparison of a geometric distance measurement between the respective abstract vectors in the common abstract vector space.

The geometric distance measurement may represent a measure of the similarity or dissimilarity between two vectors in a multi-dimensional vector space. The common abstract vector space may thereby include a high-dimensional space, in which the abstract vectors may be represented as points.

Different geometric distance measurements may be used for the similarity comparison. A possible distance measurement may be a Euclidean distance, which measures the direct distance between two points in the vector space. Alternatively, the Manhattan distance and/or the cosine similarity may be used as the distance measurement. The choice of the specific distance measurement may depend on the type of coded data and the desired similarity definition.

The geometric distance measurement may be compared by calculating the distance between the abstract vector of the first entity and each of the other abstract vectors stored in the collection. The identification data for the second entity may then be provided on the basis of the smallest calculated distance or on the basis of a ranking of the calculated distances.

Using a geometric distance measurement in a common abstract vector space may facilitate an efficient and scalable method for determining similarities between complex datasets. By representing the examination data as abstract vectors in a common space, similarities may also be detected between examination data with a different structure and/or modality.

A precise and differentiated selection of second entity may advantageously be facilitated by using a geometric distance measurement for the similarity comparison. The method may be adjusted flexibly for different applications and data types by selecting the most suitable distance measurement. Additionally, the calculation of geometric distances may be implemented efficiently in high-dimensional spaces, which may also facilitate fast processing of large amounts of data.

The disclosure relates in a second aspect to a computer-implemented method for providing a collection of abstract vectors. In one act, a multitude of examination data from imaging-based examinations of respective examination objects may be captured by respective reference entities. In another act, a trained function is applied to input data, wherein the input data is based on the multitude of examination data. A multitude of abstract vectors is thereby provided in a common abstract vector space. The trained function is also based on an abstract vector model. In another act, the multitude of abstract vectors is collected in a data structure, wherein identification data for the respective reference entity is assigned to each of the abstract vectors in the data structure. The collection of abstract vectors is provided in another act.

The advantages of the proposed computer-implemented method for providing a collection of abstract vectors may correspond to the advantages of the proposed method for providing identification data for a second entity. The features, advantages, or alternative embodiments mentioned here may also be transferred to the other claimed subject matters and vice versa.

Capturing the multitude of examination data may include recording and/or receiving the examination data. The examination data may be captured by a sensor and/or a medical imaging device. The imaging-based examinations may include different medical imaging procedures, such as, for example, X-rays, CT, MRI, ultrasound, and/or PET.

The examination data, the examination objects, and the identification data may advantageously have all the features and properties of the examination data, the examination object, and the identification data that were described in relation to the method for providing identification data for a second entity and vice versa.

The reference entities may be medical entities, medical facilities, competence centers, doctors, medical specialists, and/or technical specialists, which perform the imaging-based examinations for providing the examination data.

The trained function may be a function that was trained on a set of training data through machine learning. The abstract vector model, on which the trained function is based, may be a mathematical model that represents complex data in a high-dimensional vector space. The common abstract vector space may be a mathematical space, in which the abstract vectors are comparable to the different items of examination data. Advantageously, the trained function may be provided by a proposed method for providing a trained function.

Collecting the multitude of abstract vectors in a data structure may include storage in a data memory unit, a database, and/or a cloud. The data structure may be designed so that it facilitates efficient storage and retrieval of the abstract vectors and their assigned identification data.

The identification data for the respective reference entity may include information that facilitates a clear identification of the respective reference entity. Assigning the identification data for the respective reference entity to the abstract vectors may include storage in pairs in a common data structure. Alternatively, or additionally, an assignment table may be used, in which the identification data for the corresponding reference entity is assigned to each of the abstract vectors.

Providing the collection of abstract vectors may include storage on a computer-readable storage medium, and/or displaying on a display unit, and/or transferring to a provision unit.

Providing a collection of abstract vectors may facilitate efficient processing and analysis of large amounts of examination data. The abstract vectors may serve as compact representations of the original examination data, which may reduce memory requirements and/or increase processing speed. Additionally, assigning identification data to the abstract vectors may facilitate a fast and targeted search for relevant reference entities.

In another advantageous embodiment of the computer-implemented method for providing a collection of abstract vectors, the examination data may include at least one of the following elements: medical image data of the respective examination object, medical diagnostic data of the respective examination object, medical anamnesis data of the respective examination object, recording parameters of a medical imaging device, operating parameters of a medical object during an imaging-based examination, measured values of a medical monitoring device, a language of the medical diagnostic data and/or of the anamnesis data of the respective examination object, metadata of the medical image data, and/or of the medical diagnostic data, and/or of the anamnesis data, and/or location information for the reference entity.

The medical image data of the respective examination object, medical diagnostic data of the respective examination object, medical anamnesis data of the respective examination object, recording parameters of a medical imaging device, operating parameters of a medical object during an imaging-based examination, measured values of a medical monitoring device, the language of the medical diagnostic data and/or of the anamnesis data of the respective examination object, the metadata of the medical image data, and/or of the medical diagnostic data, and/or of the anamnesis data, and/or location information for the reference entity may advantageously have all the features and properties of the examination data and medical image data of the examination object, medical diagnostic data of the examination object, medical anamnesis data of the examination object, recording parameters of a medical imaging device, operating parameters of a medical object during an imaging-based examination, measured values of a medical monitoring device, the language of the medical diagnostic data and/or of the anamnesis data of the examination object, the metadata of the medical image data, and/or of medical diagnostic data, and/or of the anamnesis data, and/or location information for the first entity, which were described in relation to the method for providing identification data for a second entity and vice versa.

Incorporating this varied examination data when creating abstract vectors may facilitate a comprehensive and nuanced representation of the medical cases. By taking different data types into account, the method may potentially achieve improved accuracy and relevance when identifying similar cases or choosing suitable second entities. The variety of the data may also help to cover a broader range of medical scenarios and thus increase the applicability and flexibility of the system.

The examination data may advantageously also include a keyword list. This keyword list may be generated automatically on the basis of the other, particularly the remaining, types of examination data. For example, a keyword list may be created automatically from identified image content on the imaging device or in an image database of the reference entity. This keyword list may serve as a profile for the reference entity. Additionally, findings reports may be aggregated and automatically given keywords, in order to expand the keyword list. Moreover, the reference entity may have the opportunity to access the keyword list manually. The reference entity may supplement or correct the keyword list. This manual processing may enable highlighting of specific aspects of the case, which may not have been captured automatically, and/or removal of irrelevant keywords.

In another advantageous embodiment of the computer-implemented method for providing a collection of abstract vectors, the reference entities may include medical facilities, competence centers, doctors, medical specialists, and/or technical specialists.

The reference entities may capture, receive, and/or provide the examination data that is used to provide the abstract vectors. Medical facilities may include hospitals, clinics, medical practices, and/or specialized diagnostic centers. These facilities may have the necessary infrastructure and expertise to perform imaging-based examinations and to capture the corresponding examination data.

Competence centers may include specialized facilities that focus on certain medical fields or pathologies. These centers may supply particularly valuable and detailed examination data due to their specialization, which may help to improve the quality and variety of the abstract vectors. The medical competence centers may include a cluster, which includes a group of experts and/or a specialized department. A cluster may provide bundled expertise for certain medical issues, thereby helping to generate more precise and meaningful abstract vectors.

As individual reference entities, doctors may incorporate their personal expertise and experience into the capture and interpretation of the examination data. This may lead to a variety of perspectives and approaches in the collected examination data, which may increase the robustness and applicability of the resulting abstract vectors.

The reference entity may also include medical specialists, for example, radiology technicians and/or specialist nurses. These medical specialists may make a valuable contribution to the quality and consistency of the captured examination data due to their practical experience in performing imaging-based examinations.

The reference entity may also include technical specialists, such as personnel who operate the medical imaging device, and/or medical objects, and/or monitoring devices, and/or who use medical devices. These technical specialists may make a valuable contribution to the quality and consistency of the captured examination data due to their practical experience in operating and/or using medical imaging devices, medical objects, monitoring devices, and/or medical products.

Incorporating different types of reference entities may lead to a comprehensive and varied collection of examination data. This may improve the representativeness and applicability of the abstract vectors derived therefrom. The variety of reference entities may also help to reduce potential biases in the data and to provide a broader coverage of different medical scenarios and patient groups. By using different types of reference entities, the method may advantageously provide a more comprehensive and balanced collection of abstract vectors. This may lead to improved performance in identifying similar cases and providing relevant identification data.

In another advantageous embodiment of the computer-implemented method for providing a collection of abstract vectors, collecting the multitude of abstract vectors may include storage in a data memory unit, a database, and/or a cloud.

Storing the abstract vectors in a data memory unit may include local storage on an individual computer system or a network of computer systems. A data memory unit may be a hard disk, a solid-state drive or a RAID system (redundant array of independent disks). Using a local data memory unit may facilitate fast access to the stored abstract vectors with the respectively assigned identification data, as no network latency occurs when transferring data.

Storing in a database may include using a data management system, which facilitates efficient organization, management and retrieval of the abstract vectors with the respectively assigned identification data. A database may be relational or non-relational (NoSQL). Relational databases may be particularly suitable if the abstract vectors with the respectively assigned identification data are to be stored in table form and complex queries are required. Non-relational databases may be advantageous if high scalability and flexibility in the data structure are required.

Storage in the cloud may include using cloud computing services, with which the abstract vectors with the respectively assigned identification data are stored on distributed servers in data centers. Cloud storage may facilitate high scalability, redundancy, and geographic distribution of the data. This may be particularly advantageous if a large number of abstract vectors need to be stored with the respectively assigned identification data or if global access to the data is required. In particular, access to the collection of abstract vectors with the respectively assigned identification data may be facilitated for multiple first entities, for example, institutes and/or doctors, in a common network. Furthermore, the collection of abstract vectors with the respectively assigned identification data for the reference entities may be stored locally in a respective data structure for different units, particularly the reference units and/or groups of multiple reference entities, for example, institutes.

The choice of storage solution may depend on various factors, such as the amount of data to be stored, the access speed requirements, the necessary scalability, the security requirements, and cost considerations. For example, a combination of local data storage for frequently used data and cloud storage for archiving and backup may be used.

Storing the abstract vectors with the respectively assigned identification data in different storage systems may facilitate improved data security through redundancy. Storing copies of the abstract vectors with the respectively assigned identification data in different systems may minimize the risk of data loss.

Furthermore, storing in different systems may facilitate load balancing, which means that queries of the collection of abstract vectors with the respectively assigned identification data may be distributed to different storage systems, improving overall performance.

The choice of storage system may also affect the implementation of the similarity comparison. For example, specialized database systems may be used for vector data, which offer optimized algorithms for the similarity comparison in the high-dimensional vector space.

Due to the flexibility in the choice of storage system, the computer-implemented method for providing a collection of abstract vectors may be adjusted to different application scenarios and requirements. This may facilitate efficient and scalable implementation of the method in different environments.

In another advantageous embodiment of the computer-implemented method for providing a collection of abstract vectors, the method may also include updating the collection of abstract vectors by repeatedly adding new abstract vectors with respectively assigned identification data on the basis of newly captured examination data. The updating may be effected periodically or continually in particular.

Repeatedly adding new abstract vectors with respectively assigned identification data advantageously allows for the collection of abstract vectors with the respectively assigned identification data to be kept up-to-date at all times. New examination data may be captured in the process, for example, from new imaging-based examinations that were performed after the initial creation of the collection of abstract vectors with the respectively assigned identification data. This newly captured examination data may then be converted into new abstract vectors by the trained function and added to the existing collection. The identification data for the respective reference entity may thereby be assigned to the newly provided abstract vectors.

Periodic updating may be effected at regular intervals, such as daily, weekly, or monthly. In contrast, continual updating may mean adding new abstract vectors with the respectively assigned identification data on a rolling basis as soon as new examination data is available.

Regularly updating the collection of abstract vectors with the respectively assigned identification data provides that the collection contains the latest available data. This may be advantageous for improving the accuracy and relevance of the identification data, which is provided on the basis of a similarity comparison with the abstract vectors stored in the collection.

Updating the collection of abstract vectors with the respectively assigned identification data may also help to capture new developments and trends in imaging-based examinations. For example, new examination techniques, imaging modalities, and/or medical findings that are reflected in the newly captured examination data may be integrated into the collection through updating. Additionally, repeatedly adding new abstract vectors may increase the variety and representativeness of the data within the collection. This may be particularly advantageous for capturing rare or new types of cases and thus improving the system's capability of providing suitable identification data even for unusual or complex examination situations.

The disclosure relates in a third aspect to a computer-implemented method for providing a trained function. In one act, training examination data for a multitude of imaging-based training examinations of one or more training entities may be provided, particularly on the basis of or by way of one or more training entities. In another act, the trained function is applied to input data, which is based on the training examination data. A respective abstract training vector is thereby provided. In another act, at least one parameter of the trained function is adjusted on the basis of the training examination data and the abstract training vector. The at least one parameter of the trained function may be adjusted by a supervised coding regression or a self-supervised adjustment of a coder-decoder structure. The trained function is provided in another act.

The advantages of the proposed computer-implemented method for providing a trained function may correspond to the advantages of the proposed method for providing identification data for a second entity and/or a method for providing a collection of abstract vectors. The features, advantages, or alternative embodiments mentioned here may also be transferred to the other claimed subject matters and vice versa.

Providing the training examination data may include receiving, capturing, and/or simulating the training examination data on the basis of or by way of the respective training entity. The training examination data and the training entity may advantageously have all the features and properties of the examination data and the first entity, which were described in relation to the method for providing identification data for a second entity and/or to the method for providing a collection of abstract vectors and vice versa. In particular, the training examination data for an imaging-based examination of one or more training examination objects may be provided, wherein the training examination data has been captured by the training entity and/or has been simulated on the basis of the training entity. The training examination objects may thereby advantageously have all the features and properties of the examination object, which were described in relation to the method for providing identification data for a second entity and/or the method for providing a collection of abstract vectors and vice versa.

The trained function may include a machine-learning model that is designed to transform input data, particularly the training examination data, into an abstract vector space. Applying the trained function to input data that is based on the training examination data provides one abstract training vector in each instance, particularly as output data of the trained function. In particular, the input data for the trained function may include the training examination data. The abstract training vector may be understood as a compressed representation of the main features of the training examination data.

Adjusting the at least one parameter of the trained function may help to improve the performance of the trained function. With the supervised coding regression, the provided abstract training vectors may be compared with known target values, and the parameters of the function may be adjusted to minimize any deviation between the abstract training vectors and the known target values. With the self-supervised adjustment of a coder-decoder structure, the trained function may be adjusted so that it may reconstruct the input data as accurately as possible without explicit target values being predefined.

Providing the trained function in the subsequent act may make the adjusted, in particular optimized, trained function available for other applications. This trained function may be used in the method described in the first aspect for providing identification data for a second entity and/or in the method described in the second aspect for providing a collection of abstract vectors.

By iteratively adjusting the trained function on the basis of a multitude of training examination data, the accuracy and robustness of the function may be improved. This may lead to more precise mapping of the examination data in the abstract vector space, which may in turn increase the quality of the identification of similar cases.

In certain examples, a trained function imitates cognitive functions that humans associate with a human brain. In particular, the trained function is able to adapt to new situations and to detect and extrapolate patterns through training based on training data. Another term for “trained function” is “trained model.” In certain examples, the parameters of a trained function may be adjusted by training. In particular, monitored training, semi-monitored training, unmonitored training, reinforcement learning, and/or active learning may be used. Representation learning (or “feature learning”) may also be used. In particular, the parameters of the trained function may be adapted iteratively through multiple training acts. In particular, a specific cost function may be minimized within the training. In particular, the backpropagation algorithm may be used within the training of a neural network structure. In particular, a trained function may include a neural network, a support vector machine, a decision tree, and/or a Bayesian network, and/or the trained function may be based on k-means clustering, Q-learning, genetic algorithms, and/or association rules. In particular, a neural network may be a deep neural network, a convolution-based neural network, or a convolution-based deep neural network. Furthermore, a neural network may be an adversarial network, a deep adversarial network, and/or a generative adversarial network.

A convolution-based neural network is a neural network that uses a convolution operation in at least one of its layers (so-called “convolution layer”) instead of a general matrix multiplication. In particular, a convolution layer performs a scalar product of one or more convolution kernels with the input data/images of the convolution layer, wherein the entries of the one or more convolution kernels are the parameters or weights that are adjusted through training. In particular, the Frobenius scalar product and the ReLU activation function may be used. A convolution-based neural network may include additional layers, e.g., pooling layers, fully bound layers, and normalization layers.

By using convolution-based neural networks, medical image data may be processed very efficiently as input data, for example, as a convolution operation based on different kernels may extract different image features so that the relevant image features may be found during training by adjusting the weights of the convolution kernel. Moreover, fewer parameters need to be trained due to the weight distribution in the convolution kernels, which prevents overfitting in the training phase and facilitates faster training or more layers in the network, whereby the network's performance is improved.

According to one aspect, the trained function may include one or more residual networks (ResNets). In particular, a ResNet is an artificial neural network that includes at least one jump or skip connection, which is used to skip at least one layer of the artificial neural network. In particular, a ResNet may be a convolution-based neural network that includes one or more skip connections, which each skip one or more convolution layers. According to some examples, the ResNets may be represented as m-layer ResNets, wherein m is the number of layers in the corresponding architecture and, according to some examples, may take values of 34, 50, 101, or 152. According to some examples, such an m-layer ResNet may include (m−2)/2 skip connections in each case.

A skip connection may be seen as a bypass, which transmits the output of a preceding layer directly over one or more skipped layers to one of the layers following the one or more skipped layers. Instead of having to adjust the desired mapping directly, the skipped layers would then have to adjust residual mapping, which “balances” the directly transmitted output.

It is mathematically easier to optimize the adjustment of the residual mapping than the direct mapping. Moreover, this mitigates the problem of vanishing/exploding gradients during optimization when training the trained function: If a skipped layer encounters such problems, its contribution may be skipped by regulating the directly transmitted output. Using ResNets thus has the advantage that much deeper networks may be trained. In the context of the intended provision of abstract vectors on the basis of examination data, this has the advantage of more accurate forecasting of the abstract vectors, thereby improving identification of suitable second entities.

A generative adversarial model (or GA model) includes a generative function and a discriminant function, wherein the generative function produces synthetic data and the discriminant function discriminates between synthetic and real data. By training the generative function and/or the discriminant function, on the one hand, the generative function is configured to produce synthetic data, which is incorrectly classified as real by the discriminant function. On the other hand, the discriminant function is configured to discriminate between real data and synthetic data produced by the generative function. In terms of game theory, a generative adversarial model may be interpreted as a zero-sum game. Training the generative function and/or the discriminant function is based in particular on minimizing a cost function.

By using a GA model, synthetic data may be produced on the basis of a set of training data, which has the same properties as the training dataset. The GA model may be trained on the basis of unannotated data (unsupervised learning) so that the effort required to train a GA model is low.

In particular, a recurrent trained function is a trained function whose output depends not only on the input value and the parameters adjusted by the training process, but rather also on a hidden state vector, wherein the hidden state vector is based on previous inputs that were used for the recurrent trained function. In particular, the recurrent trained function may include additional memory states or additional structures that incorporate time delays or include feedback loops.

In particular, the underlying structure of a recurrent trained function may be a neural network, which may be referred to as a recurrent neural network. Such a recurrent neural network may be described as an artificial neural network, in which the connections between nodes form a directed graph along a chronological sequence. In particular, a recurrent neural network may be interpreted as a directed acyclic graph. In particular, the recurrent neural network may be a recurrent neural network with finite impulse response or a recurrent neural network with infinite impulse response (wherein a network with finite impulse response deconvolves and may be replaced by a strictly feed-forward neural network, and a network with infinite impulse response does not deconvolve and may be replaced by a strictly feed-forward neural network).

In particular, a recurrent neural network may be trained on the basis of the BPTT (backpropagation through time) algorithm, the RTRL (real-time recurrent learning) algorithm, and/or on genetic algorithms.

By using a recurrent trained function, input data with sequences of variable length may be used. In particular, this means that the method may be used not only for a fixed number of input datasets (and has to be trained differently for any other number of input datasets used as input), but may also be used for any number of input datasets. This means that the entire set of training data, regardless of the number of input datasets contained in different sequences, may be used within the training and that training data may not be reduced to training data that corresponds to a specific number of sequential input datasets.

A transformer network is a neural network architecture that may include an encoder, a decoder, or both an encoder and a decoder. In some cases, the encoder and/or decoder includes multiple corresponding encoding layers and decoding layers. There may be an attention mechanism within each encoding and decoding layer. The attention mechanism, sometimes referred to as self-attention, connects data elements (such as words or pixels) within a series of data elements to other data elements within this series. The self-attention mechanism enables the model, for example, to examine a group of voxels within a medical image and to determine the relative significance of other groups of voxels within this medical image for the examined group of voxels.

The encoder may be configured in particular to transform the input into a numeric representation. The numeric representation may include one vector per input token. The encoder may be configured to implement an attention mechanism so that each vector of a token is influenced by the other tokens in the input. In particular, the encoder may be configured so that the representations resolve the desired output of the transformer network. In particular, the trained function may be designed as autoencoder-based architecture. A latent space may thereby be interpreted as an abstract vector space.

The decoder may be configured in particular to transform an input into a sequence of output tokens. In particular, the decoder may be configured to implement a masked self-attention mechanism so that each vector of a token is only influenced by the other tokens on a page of a sequence.

Furthermore, the decoder may be autoregressive, which means that interim results are returned.

According to some examples, the decoder input is based on the encoder output or is equivalent to the encoder output.

Furthermore, the transformer network may include a classification module that is configured to map the encoder output to a number of learned outputs.

According to some examples, the transformer model may be trained in two phases, a pre-training phase and a fine-tuning phase. In the pre-training phase, a transformer model may be trained on a large body of data in order to learn the underlying semantics of the problem. Such pre-trained transformer models are available for different languages. For certain applications described here, the fine-tuning may include additional training of the transformer network using medical texts with meanings annotated by experts and/or medical ontologies like RADLEX and/or SNOMED. According to some examples, with the latter the transformer model may learn relationships and synonyms for medical expressions in particular.

One advantage of transformer networks is that transformer networks may efficiently handle long-range dependencies in input data due to the attention mechanism. Moreover, the encoders used in transformer networks are able to process data in parallel, which saves computing resources for the inference. Moreover, decoders of transformer networks are able to generate a sequence of output tokens iteratively with great confidence due to the autoregression.

The different network architectures and training methods may be used to adjust and optimize the trained function on the basis of the training examination data. The use of advanced techniques like ResNets, generative adversarial models, and/or transformer networks may improve the performance and accuracy of the trained function when processing examination data and generating abstract vectors.

The trained function may also be trained using classic pattern descriptors like SIFT (scale-invariant feature transform), SURF (speeded up robust features), or FAST (features from accelerated segment test). These algorithms may be used to extract and describe characteristic features from the training examination data.

The SIFT algorithm may identify local features in the training examination data, which are invariant in terms of scaling, rotation, and partially in terms of lighting changes. The SIFT algorithm may first recognize key points in the image data and then calculate a descriptor for each key point that describes the local image structure. These descriptors may be provided as an abstract comparative vector.

The SURF algorithm, which was developed as a faster alternative to SIFT, may also be used to extract features from the training examination data. SURF uses integral images and a simplified version of the Hessian matrix to identify key points and calculate descriptors. The resulting SURF descriptors may be provided as an abstract comparative vector.

The FAST algorithm may be used to detect vertices quickly in the training examination data. FAST compares the intensity of a pixel with the surrounding pixels to identify vertices. The detected vertices may serve as the basis for creating abstract comparative vectors.

The classic algorithm may be used to generate abstract comparative vectors, which are compared with the abstract training vector. The trained function, particularly at least one parameter of the trained function, may be adjusted on the basis of this comparison. Advantageously, the at least one parameter of the trained function may be adjusted in such a way that deviation between the abstract comparative vector and the abstract training vector is minimized.

By using abstract vectors, particularly embedding vectors, frequently occurring patterns may advantageously be coded in the trained function during training. These patterns may include contrasted vascular trees, movement sequences of medical instruments, and/or unknown medical instruments. When the first entity, for example, a doctor seeking advice, uses a method for providing identification information for a second entity, the current examination data, for example, medical image data, may be mapped in an abstract vector and compared with these coded patterns. An advantage of this approach is that there is no need to create a database manually of procedures, anatomies, and instruments that would have to be pre-trained in the networks. Instead, the trained function may learn the relevant patterns and structures independently during the training process and embed these in the abstract vector space. This may improve the flexibility and adjustability of the trained function, as it is able to detect and process new types of patterns or unexpected patterns in the training examination data.

According to one advantageous embodiment, the trained function may be embodied as a large language model (LLM). An LLM may be an advanced machine-learning model, which has been trained on large amounts of text data and is able to handle complex language tasks. The LLM may be used as a specific form of trained function, in order to process input data on the basis of the examination data, particularly the training examination data during the training, and to provide the abstract vector as output data. The LLM may thereby be designed for LLM embedding of the examination data. In this configuration, the LLM may process the input data on the basis of the examination data, such as, for example, medical image data, findings, and/or anamneses. This examination data may be in natural language and/or in structured form.

The LLM may analyze the input data and extract relevant information. On the basis of this analysis, the model may generate tagging for the examination data. This tagging may be seen as a type of abstract vector representing the key features and concepts of the examination data.

The generated tagging may then be used to perform a subject search. This subject search may include both text-based and image-based elements. The LLM may access a comprehensive knowledge base, which not only includes the collection of abstract vectors but also freely available databases, such as scientific publications, published case reports, and/or conference videos. On the basis of this expanded search, the LLM may generate literature suggestions and subject suggestions for research of professional articles. These suggestions may be provided as part of the identification data for the second entity. Moreover, the LLM may identify other potential experts as second entities using the author lists for the suggested literature and the cited references.

A technical advantage of this LLM-based approach lies in its ability to generate context-specific and highly relevant identification data for the second entities. The LLM may detect complex relationships in the examination data and correlate these to a broad range of specialist medical knowledge.

Additionally, the LLM may evaluate current medical image data in particular during an ongoing intervention and may suggest a context for identifying the second entity, particularly the expert request. This may include problems with stents, questions about anatomy, complications, or questions about the imaging device. This dynamic approach facilitates continual adjustment and refinement of the search for suitable second entities based on the current progress of the examination. The context for identifying the second entity may be used in the similarity comparison, in order to identify relevant second entities. The context for identifying the second entity may thereby be considered an additional dimension in the abstract vector space. This means that not only the examination data but also the specific context of the request may be taken into account in the similarity comparison. For example, second entities with expertise in specific problems, anatomical structures, or imaging modalities may be provided, which correspond to the context of the expert request. The similarity comparison may thus be adjusted dynamically to the actual progress of the examination and the specific issues. This may lead to more precise selection of suitable second entities, which have not only handled similar image content but also have experience of comparable clinical issues.

In another advantageous embodiment of the computer-implemented method for providing a trained function, the training examination data may include at least one of the following elements: medical image data of a respective training examination object, medical diagnostic data of the respective training examination object, medical anamnesis data of the respective training examination object, recording parameters of a medical imaging device, operating parameters of a medical object during an imaging-based examination, measured values of a medical monitoring device, a language of the medical diagnostic data and/or of the anamnesis data of the respective training examination object, metadata of the medical image data, and/or of the medical diagnostic data, and/or of the anamnesis data, and/or location information for the training entity.

The medical image data of the respective training examination object, medical diagnostic data of the respective training examination object, medical anamnesis data of the respective training examination object, recording parameters of a medical imaging device, operating parameters of a medical object during an imaging-based examination, measured values of a medical monitoring device, the language of the medical diagnostic data and/or of the anamnesis data of the respective training examination object, the metadata of the medical image data, and/or of the medical diagnostic data, and/or of the anamnesis data, and/or location information for the training entity may advantageously have all the features and properties of the medical image data of the examination object, medical diagnostic data of the examination object, medical anamnesis data of the examination object, recording parameters of a medical imaging device, operating parameters of a medical object during an imaging-based examination, measured values of a medical monitoring device, the language of the medical diagnostic data and/or of the anamnesis data of the examination object, the metadata of the medical image data, and/or of the medical diagnostic data, and/or of the anamnesis data, and/or location information for the first entity, which were described in relation to the method for providing identification data for a second entity and vice versa.

By incorporating this varied training examination data, the trained function may advantageously be designed to map the training examination data more comprehensively and precisely to an abstract (training) vector. This may lead to improved performance in the analysis and interpretation of examination data. Moreover, taking different data types into account may increase the trained function's robustness and adjustability to different clinical scenarios. Additionally, the trained function may be designed to differentiate depending on an activity of the involved specialist, for example, a doctor, as an additional dimension of the training vector and/or a scaling of the training vector.

In another advantageous embodiment of the computer-implemented method for providing a trained function, adjusting the at least one parameter of the trained function may include a federated adjustment of the at least one parameter of the trained function.

The federated adjustment, also referred to as federated learning, may be a method for machine learning in which a model is trained via multiple local devices or servers, without exchanging local training data. This concept may be particularly useful if data protection and confidentiality are vitally important, as may be the case in the medical field.

With a federated adjustment method, multiple entities or facilities may each train local copies of the trained function on their own data. Instead of sharing raw data, just the parameter updates or gradient information may be sent to a central server or coordinator. This may then aggregate the contributions from all participants and update a global model.

Federated adjustment may offer multiple advantages. First, it may protect the private sphere, as sensitive data does not leave the local systems. Second, it may increase efficiency by reducing the necessity for large data transfers. Third, it may improve the robustness of the model by learning from a broader and more varied database.

In the context of the proposed method for providing a trained function, federated adjustment may be implemented in such a way, for example, that different medical facilities train their own local versions of the trained function on their specific training examination data. The resulting parameter updates may then be sent to a central coordinating body, which amalgamates this information and creates an improved global model.

This global model may then be distributed to the participating facilities, where it may serve as the starting point for the next round of training. This iterative process may be continued until a desired performance or convergence is achieved.

Federated adjustment may enable the trained function to profit from a broader range of data and experiences, without sensitive patient data having to be exchanged between facilities. This may lead to a more robust and accurate trained function, which is better able to handle different clinical scenarios.

By implementing a federated adjustment approach, the proposed method may advantageously improve the quality and generalizability of the trained function, while simultaneously protecting the confidentiality of the patient data.

The disclosure relates in a fourth aspect to a provision unit. The provision unit may be designed to execute a proposed computer-implemented method for providing identification data for a second entity and/or a proposed computer-implemented method for providing a collection of abstract vectors.

The advantages of the proposed provision unit may correspond to the advantages of the proposed method for providing identification data for a second entity and/or a method for providing a collection of abstract vectors. The features, advantages or alternative embodiments mentioned here may also be transferred to the other claimed subject matters and vice versa.

The provision unit may advantageously include a computing unit, a memory unit, and/or an interface. The provision unit, particularly the components of the provision unit, may be designed to execute the individual acts of the proposed method.

The interface of the provision unit may be designed to capture examination data from an imaging-based examination of an examination object by a first entity. Furthermore, the computing unit and/or the memory unit of the provision unit may be designed to provide an abstract vector by applying a trained function to input data, which is based on the examination data of the first entity. The computing unit and/or the memory unit may also be designed to provide identification data for a second entity on the basis of a similarity comparison of the abstract vector with other abstract vectors in a common abstract vector space stored in a collection.

Furthermore, the provision unit may be designed to capture a multitude of examination data from imaging-based examinations of respective examination objects by respective reference entities, to apply a trained function to input data, which is based on the multitude of examination data, wherein a multitude of abstract vectors is provided in a common abstract vector space, to collect the multitude of abstract vectors in a data structure, wherein identification data for the respective reference entity is assigned to each of the abstract vectors in the data structure, and to provide the collection of abstract vectors.

The interface of the provision unit may be designed to capture the multitude of examination data. The computing unit may be designed to apply the trained function to the input data, which is based on the multitude of examination data, wherein the multitude of abstract vectors is provided. The memory unit may be designed to collect the multitude of abstract vectors with the respectively assigned identification data for the reference entity in the data structure, wherein identification data for the respective reference entity is assigned to each of the abstract vectors in the data structure. The interface may also be designed to provide the collection of abstract vectors.

The provision unit may advantageously be configured so that it may efficiently and reliably execute all acts and functions of the proposed computer-implemented method for providing identification data for a second entity and/or the proposed computer-implemented method for providing a collection of abstract vectors. Improved processing and analysis of imaging-based examination data may be facilitated by using such a provision unit.

The disclosure relates in a fifth aspect to a system including a medical imaging device and a proposed provision unit. The medical imaging device is designed to capture medical image data of the examination object and to provide it to the provision unit.

The medical imaging device may include different modalities, such as an X-ray device, a CT device, an MRI device, an ultrasound device, and/or a nuclear medicine imaging device, for example, a PET device.

The provision unit may be designed to receive the medical image data provided by the medical imaging device and to process it as examination data of the imaging-based examination of the examination object. The processing may include applying the trained function to input data, which is based on the medical image data, in order to provide an abstract vector.

The interaction between the medical imaging device and the provision unit may be effected via a suitable interface. The interface may include a wired or wireless data connection. Data transfer may be effected in real time or asynchronously.

The advantages of the proposed system may correspond to the advantages of the proposed method for providing identification data for a second entity and/or a method for providing a collection of abstract vectors. The features, advantages or alternative embodiments mentioned here may also be transferred to the other claimed subject matters and vice versa.

The disclosure relates in a sixth aspect to a (non-transitory) computer program product or computer-readable medium having a computer program, which may be loaded directly into a memory of a computer, with program sections to execute a proposed computer-implemented method for providing identification data for a second entity, a proposed computer-implemented method for providing a collection of abstract vectors, and/or a proposed computer-implemented method for providing a trained function, if the computer program is executed by the computer.

The computer program product may include software with a source code that still needs to be compiled and bound or that only needs to be interpreted, or an executable software code that only needs to be loaded into the computer for execution. By a computer, the computer program product may execute the proposed method quickly and robustly and may reproduce it identically. The computer program product may be configured so that it may execute the method acts by the computer.

The computer program product may be saved to a computer-readable storage medium or be stored on a network or server, from where it may be loaded onto the processor of a computer, which may be directly linked to the computer or be designed as part of the computer. Control information of the computer program product may also be stored on an electronically readable data carrier. The control information of the electronically readable data carrier may be arranged in such a way that it executes an method when the data carrier is used in a computer. Examples of electronically readable data carriers may include a DVD, a magnetic tape, or a USB stick, on which electronically readable control information, in particular software, is stored. If this control information is read from the data carrier and stored in a computer, all embodiments of the methods described above may be executed.

An advantage of a software-based implementation may be that previously used computers may also be easily retrofitted through a software update, in order to work in the manner according to the disclosure. In addition to the computer program, such a computer program product may include, where necessary, additional elements such as, for example, documentation and/or additional components, as well as hardware components such as, for example, hardware keys (dongles etc.) for using the software.

The computer program product may advantageously be configured so that it directs the computer to provide an abstract vector by applying a trained function to input data, which is based on examination data of a first entity. Furthermore, the computer program product may direct the computer to provide identification data for a second entity on the basis of a similarity comparison of the abstract vector with other abstract vectors in a common abstract vector space stored in a collection.

The computer program product may also direct the computer to provide a collection of abstract vectors, by capturing a multitude of examination data from imaging-based examinations, applying a trained function to input data, which is based on the multitude of examination data, wherein a multitude of abstract vectors is provided, and by collecting the multitude of abstract vectors in a data structure, wherein identification data for the respective reference entity is assigned to each of the abstract vectors in the data structure.

The computer program product may also direct the computer to provide a trained function, by providing training imaging data from a multitude of imaging-based training examinations, applying the trained function to input data, which is based on the training examination data, and adjusting at least one parameter of the trained function on the basis of the training examination data and the abstract training vector by a supervised coding regression or a self-supervised adjustment of a coder-decoder structure.

The computer program product may combine the advantages of the proposed methods and of the computer in a flexible and adjustable software solution. By implementing it as a computer program product, the disclosure may be used on various hardware platforms and in different system environments, which may facilitate broad applicability and simple integration in existing systems.

The disclosure may also relate to a training unit, which is configured to execute a proposed method for providing a trained function. The training unit may thereby advantageously include a training interface, a training memory unit, and/or a training computing unit. The training unit may be designed to execute a method for providing a trained function, by designing the training interface, the training memory unit, and/or the training computing unit to execute the corresponding method acts.

Advantageously, the training interface may be designed to capture training examination data from a multitude of imaging-based training examinations of one or more training entities. Furthermore, the training computing unit and/or the training memory unit may be designed to apply the trained function to input data, which is based on the training examination data, wherein a respective abstract training vector is provided, and to adjust at least one parameter of the trained function on the basis of the training examination data and the abstract training vector by a supervised coding regression or a self-supervised adjustment of a coder-decoder structure.

The advantages of the proposed training unit may correspond to the advantages of the proposed computer-implemented method for providing a trained function. The features, advantages or alternative embodiments mentioned here may also be transferred to the other claimed subject matters and vice versa.

### BRIEF DESCRIPTION OF THE DRAWINGS

Exemplary embodiments of the disclosure are illustrated in the drawings and described in more detail below. The same reference signs are used for the same features in different figures.

FIGS. 1 and 2 depict schematic representations of different advantageous embodiments of a proposed computer-implemented method for providing identification data for a second entity.

FIGS. 3 and 4 depict schematic representations of different advantageous embodiments of a proposed computer-implemented method for providing a collection of abstract vectors.

FIG. 5 to 7 depict schematic representations of different advantageous embodiments of a proposed computer-implemented method for providing a trained function.

FIG. 8 depicts a schematic representation of an advantageous embodiment of a proposed provision unit.

FIG. 9 depicts a schematic representation of an advantageous embodiment of a proposed training unit.

FIG. 10 depicts a schematic representation of an advantageous embodiment of a proposed system.

### DETAILED DESCRIPTION

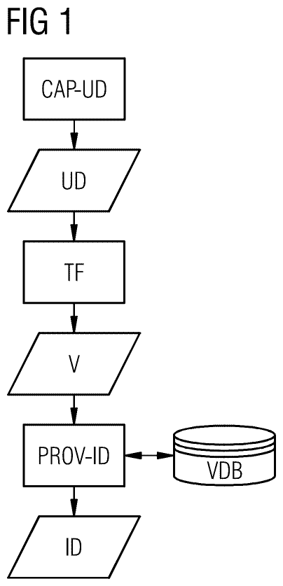

FIG. 1 shows a schematic representation of an advantageous embodiment of a computer-implemented method for providing identification data for a second entity.

The method may begin by capturing examination data CAP-UD. In this act, examination data UD from an imaging-based examination of an examination object may be captured CAP-UD by a first entity. The examination data UD may include medical imaging data, medical diagnostic data, and/or medical anamnesis data of the examination object.

In another act, a trained function TF may be applied to input data, which is based on the examination data UD. The trained function TF may be based on an abstract vector model. By applying the trained function TF to the input data, an abstract vector V may be provided. The abstract vector V may constitute a compressed representation of the examination data UD in an abstract vector space.

The method may also include providing PROV-ID identification data ID. In this act, identification data ID for a second entity may be provided PROV-ID. The provision PROV-ID of the identification data ID may be based on a similarity comparison of the abstract vector V with other abstract vectors stored in a collection VDB. The collection of abstract vectors VDB may include a database or a cloud-based memory structure, in which the abstract vectors for different reference entities are stored with assigned identification data.

The similarity comparison may be performed in a common abstract vector space. Geometric distance measurements between the abstract vector V and the stored other abstract vectors may thereby be calculated. The second entity whose abstract vector has the greatest similarity to the abstract vector V may be identified as the relevant second entity. Alternatively, or additionally, a ranking of second entities may be provided on the basis of the similarities between the abstract vector and their respective other abstract vector.

Identification information ID for the relevant second entity may be provided PROV-ID as the outcome of the method. The identification information ID may include contact data and/or expertise information for the second entity.

Using abstract vectors and the similarity comparison in a common abstract vector space facilitates an accurate selection of a relevant second entity. This may be particularly advantageous when looking for experts for a second opinion in complex medical cases.

The examination data UD may include medical image data of the examination object, medical diagnostic data of the examination object, medical anamnesis data of the examination object, recording parameters of a medical imaging device for recording medical image data of the examination object, operating parameters of a medical object during the imaging-based examination of the examination object, measured values of a medical monitoring device, a language of the medical diagnostic data and/or of the anamnesis data of the examination object, metadata of the medical image data, and/or of the medical diagnostic data, and/or of the anamnesis data, and/or location information for the first entity.

Furthermore, some of the examination data UD coded in the abstract vector V may be excluded from the similarity comparison. This means that some of the information included in the abstract vector V may be left out of the comparison with the other abstract vectors stored in the collection VDB.

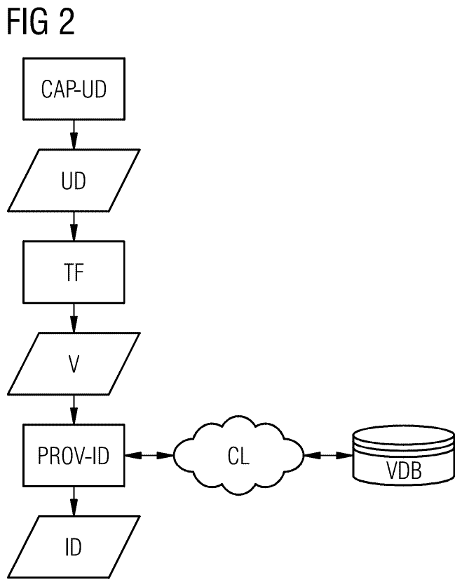

FIG. 2 shows a schematic representation of another advantageous embodiment of a computer-implemented method for providing PROV-ID identification data ID for a second entity.

The other abstract vectors with the respectively assigned identification data ID may thereby be stored in a cloud CL. When providing PROV-ID the identification data ID, the provided abstract vector V may be compared with the other abstract vectors stored in the cloud CL. This comparison may be based on a similarity measure, for example, a geometric distance in the abstract vector space. Identification data ID for a second entity may be provided PROV-ID on the basis of this comparison.

Using a cloud CL to store the collection of abstract vectors VDB may offer multiple advantages. Cloud storage may facilitate a central, easily accessible, and scalable solution for managing and accessing the collection of abstract vectors VDB. This may facilitate efficient updating and expansion of the collection, as new abstract vectors may easily be added to the cloud. Additionally, cloud storage may simplify access to the collection from different locations, which may be particularly useful if multiple entities or facilities are part of the system.

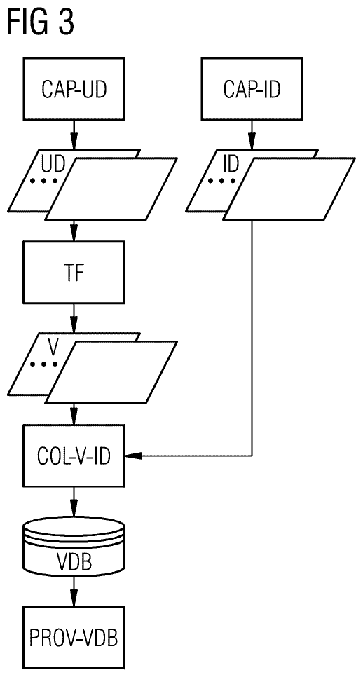

FIG. 3 shows a schematic representation of an advantageous embodiment of a computer-implemented method for providing PROV-VDB a collection VDB of abstract vectors.

The method may include capturing CAP-UD examination data UD. In this act, a multitude of examination data UD from imaging-based examinations of respective examination objects may be captured by respective reference entities. The examination data UD may include medical image data of the respective examination object, medical diagnostic data of the respective examination object, medical anamnesis data of the respective examination object, recording parameters of a medical imaging device, operating parameters of a medical object during an imaging-based examination, measured values of a medical monitoring device, a language of the medical diagnostic data and/or of the anamnesis data of the respective examination object, metadata of the medical image data, and/or of the medical diagnostic data, and/or of the anamnesis data, and/or location information for the reference entity.

Furthermore, identification data ID for the reference entity may be captured CAP-ID.

The method may also include applying a trained function TF to input data, which is based on the multitude of examination data UD. A multitude of abstract vectors V in a common abstract vector space may be provided in this act. The trained function TF may be based on an abstract vector model.

The abstract vectors may be collected COL-V-ID in another act. The multitude of abstract vectors V may be collected in a data structure in the process. Identification data ID for the respective reference entity may be assigned to each of the abstract vectors V in the data structure.

Finally, the collection VDB of abstract vectors may be provided PROV-VDB. A collection VDB of abstract vectors, which has the collected abstract vectors V with the assigned identification information ID, may be provided PROV-VDB here.

This method facilitates the provision PROV-VDB of an efficient and structured collection VDB of abstract vectors, which represent a multitude of examination data. This collection may be used advantageously for different applications, such as, for example, similarity comparisons or for identifying relevant reference entities on the basis of new examination data.

The reference entities may include medical facilities, competence centers, doctors, medical specialists, and/or technical specialists.

Furthermore, the proposed method may include updating the collection of abstract vectors VDB to provide PROV-VDB a collection VDB of abstract vectors VDB. This may be effected by repeatedly adding new abstract vectors V with respectively assigned identification data ID. The new abstract vectors may be based on newly captured examination data UD. This updating process may be performed periodically or continually, wherein new data is integrated into the existing collection VDB that is provided by the PROV-VDB act.

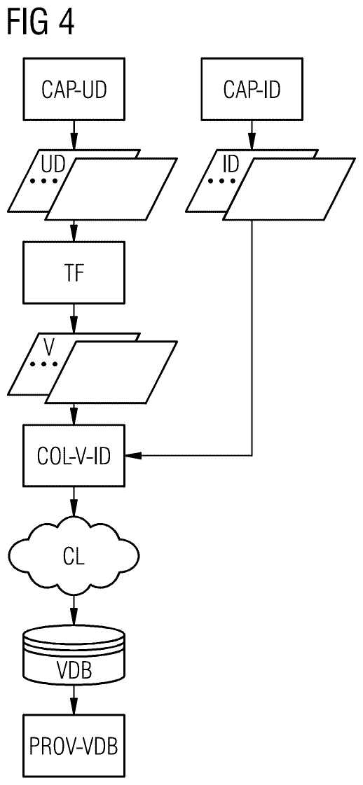

FIG. 4 shows a schematic representation of another advantageous embodiment of a computer-implemented method for providing PROV-VDB a collection VDB of abstract vectors, wherein collecting the abstract vectors includes storage in a cloud CL. The cloud CL may include a distributed computer infrastructure that is accessible via a network. An advantage of storage in the cloud CL is that the collection VDB of abstract vectors is accessible from different locations and that high scalability may be achieved.

FIG. 5 shows a schematic representation of an advantageous embodiment of a computer-implemented method for providing PROV-TF a trained function TF.

The method may begin by providing PROV-TUD training examination data TUD. The training examination data TUD that is provided in the PROV-TUD act may include at least one of the following elements: medical image data of a respective training examination object, medical diagnostic data of the respective training examination object, medical anamnesis data of the respective training examination object, recording parameters of a medical imaging device, operating parameters of a medical object during an imaging-based examination, measured values of a medical monitoring device, a language of the medical diagnostic data and/or of the anamnesis data of the respective training examination object, metadata of the medical image data, and/or of the medical diagnostic data, and/or of the anamnesis data, and/or location information for the training entity.

In another act, a trained function TF may be applied to input data, which is based on the training examination data TUD. An abstract training vector TV may be provided by this application. The abstract training vector TV may constitute a compressed representation of the training examination data TUD in a high-dimensional vector space.

The trained function TF may act in particular as an encoder, which codes the input data that is based on the training examination data TUD in an abstract training vector TV.

The abstract training vector TV may then be processed by decoder DEC. The decoder DEC may be configured to decode the abstract training vector TV and to produce a reconstruction of the original training examination data TUD.

The output of the decoder DEC may be evaluated by a cost function CF. The cost function CF may serve as a decision point in the process. If the cost function CF delivers a satisfactory result (if in the affirmative, Y), the method may proceed to providing PROV-TF the trained function TF. In this instance, the trained function TF may be provided PROV-TF.

If the cost function CF delivers no satisfactory result (if in the negative, N), the method may revert to adjusting ADJ-TF the at least one parameter of the trained function TF. Parameters for the trained function TF may be adjusted in this act in order to improve the performance of the trained function TF.

This cycle of adjusting and evaluating may continue iteratively until the criteria of the cost function CF are fulfilled. The trained function TF may be optimized in stages through this iterative process, in order to achieve the most accurate representation of the training examination data TUD. By incorporating the trained function TF, the decoder DEC, and the cost function CF, feedback may be produced that may facilitate continual improvement in the performance of the trained function TF.

Adjusting ADJ-TF the at least one parameter of the trained function TF may be viewed as a self-supervised adjustment of a coder-decoder structure. In this method, the trained function TF may act as a coder that codes the input data in an abstract vector, while the decoder DEC attempts to reconstruct the original input data from this abstract vector. The deviation between the original and the reconstructed data may then be used to adjust both the coder and the decoder, without having to specify explicit target values. In particular, the cost function CF may evaluate the deviation between the original and the reconstructed data.

Adjusting ADJ-TF the at least one parameter of the trained function TF may thereby be based on a self-supervised training of the coding-decoding network, for example, a U-Net architecture or a vision transformer architecture. The coding part of the network may then be reused as the trained function TF, particularly as an abstract vector model.

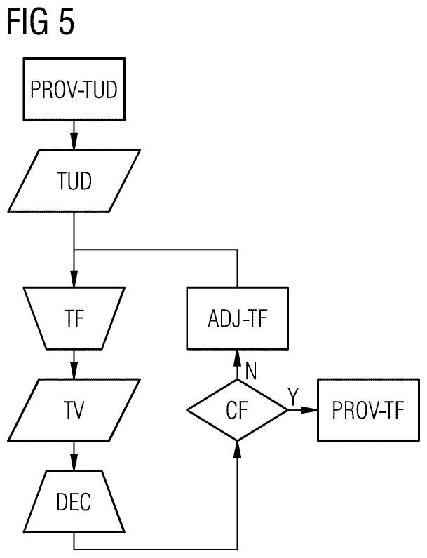

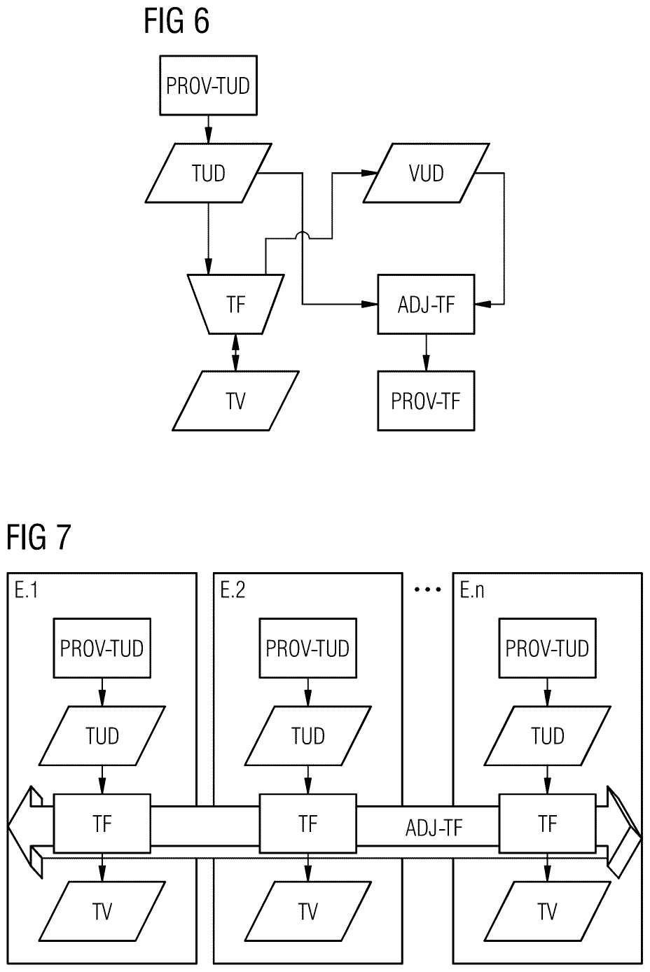

FIG. 6 shows a schematic representation of another advantageous embodiment of a computer-implemented method for providing a trained function TF.

The method may include providing PROV-TUD training examination data TUD. The trained function TF may be applied to input data, which is based on the training examination data TUD, wherein an abstract training vector TV may be provided.

Furthermore, comparative examination data VUD may be provided by reversing the trained function TF, particularly by applying the reversed trained function TF to the abstract training vector TV.

The at least one parameter of the trained function TF may be adjusted ADJ-TF on the basis of the training examination data TUD and the comparative examination data VUD. Adjusting ADJ-TF the trained function TF may include a supervised coding regression, for example, a ResNet architecture or a vision transformer architecture. Here too, the coding part of the network may be reused as the trained function TF, particularly as an abstract vector model.

After adjusting ADJ-TF the at least one parameter of the trained function TF, the trained function may be provided PROV-TF.

FIG. 7 shows a schematic representation of another advantageous embodiment of a computer-implemented method for providing a trained function TF. The method may include multiple training entities E.1, E.2 to E.n, which are involved in the training process.

Training examination data TUD may be provided PROV-TUD in one act. The training examination data TUD may be provided by a first training entity E.1, a second training entity E.2, and other training entities, where appropriate, up to an nth training entity E.n. Each training entity may constitute a separate source for training examination data TUD, for example, different medical facilities or doctors.

In another act, a trained function TF may be applied in each instance to the input data, which is based on the training examination data TUD. A respective abstract training vector TV may be provided in the process for each training entity. The abstract training vectors TV may constitute representations of the respective training examination data TUD in the abstract vector space.

The at least one parameter of the trained function TF may be adjusted ADJ-TF on the basis of the training examination data TUD and the provided abstract training vectors TV.

The at least one parameter of the trained function TF may be adjusted ADJ-TF using federated learning in multiple acts across the various training entities E.1 to E.n:

In one act, each training entity E.1 to E.n may receive a local copy of the trained function TF. These local copies may then be trained on the respective training examination data TUD of the training entities, without the raw data having to be exchanged between the training entities.

After the local training, each training entity may send the parameter updates or gradient information for their local copy of the trained function TF to a central coordinating body. This central body may be a server that has the task of aggregating the contributions from all training entities.

The central coordinating body may then amalgamate the received parameter updates, in order to create or update a global model of the trained function TF. Different aggregation methods may thereby be used, such as a weighted averaging of the parameter updates.

The updated global model of the trained function TF may then be redistributed to all training entities E.1 to E.n. This global model may then serve as the starting point for the next round of local training.

This process of local training, aggregation, and distribution of the global model may be repeated iteratively until the desired performance level is achieved or a predefined number of iterations have been performed.

This approach of federated learning means that the trained function TF may profit from the data and experience of all training entities, without the sensitive data having to be directly exchanged between the training entities. This may lead to a more robust and accurate trained function TF, which is in a better position to handle different clinical scenarios.

Additionally, federated learning may facilitate adjustment to local peculiarities. The local copies of the trained function TF may also be adjusted to the specific conditions of the respective training entity, while simultaneously profiting from the global knowledge.

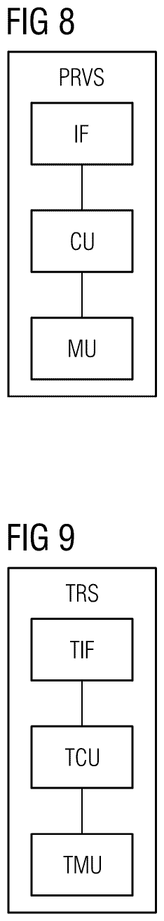

FIG. 8 shows a schematic representation of an advantageous embodiment of a provision unit PRVS. The provision unit PRVS may include an interface IF, a computing unit CU, and a memory unit MU.

The interface IF may be designed to capture examination data from an imaging-based examination of an examination object by a first entity. The interface IF may also be designed to transmit the captured examination data to the computing unit CU.

The computing unit CU may be designed to apply a trained function to input data, which is based on the examination data of the first entity. The computing unit CU may thereby provide an abstract vector. The computing unit CU may also be designed to perform a similarity comparison of the abstract vector with other abstract vectors in a common abstract vector space stored in a collection.

The memory unit MU may be designed to save the collection of stored other abstract vectors. Respective identification data may thereby be assigned to the stored other abstract vectors.

The provision unit PRVS may be designed to provide identification data for a second entity on the basis of the similarity comparison. The provision unit PRVS may thereby output the identification data for the second entity via the interface IF.

The provision unit PRVS may also be designed to execute a method for providing a collection of abstract vectors. In this instance, the components of the provision unit PRVS may be configured as follows:

The interface IF may be designed to capture a multitude of examination data from imaging-based examinations of respective examination objects by respective reference entities. The interface IF may thereby receive different types of examination data, such as medical image data, diagnostic data, or anamnesis data.

The computing unit CU may be designed to apply a trained function to input data, which is based on the multitude of examination data, wherein a multitude of abstract vectors is provided in a common abstract vector space. The computing unit CU may thereby use the trained function to transform the examination data into a compact vector representation.

The memory unit MU may be designed to collect the multitude of abstract vectors in a data structure. The memory unit MU may assign identification data for the respective reference entity to each of the abstract vectors in the data structure. The memory unit MU may use different storage technologies to facilitate efficient management and fast access to the stored data.

In some instances, the provision unit PRVS may be designed to provide the collection of abstract vectors. This may be effected, for example, by transmitting the collection to other systems or by providing access to the collection.

Additionally, the provision unit PRVS may be designed in some embodiments to update the collection of abstract vectors. The interface IF may thereby capture new examination data continually or at regular intervals. The computing unit CU may process this new data to provide new abstract vectors, which may then be integrated in the existing collection by the memory unit MU.

The components of the provision unit PRVS may be configured in some aspects so that they facilitate distributed processing and storage of the data. This may be particularly advantageous if large amounts of data need to be processed, or if the data also needs to be accessible from different locations.

The components of the provision unit PRVS may be connected to each other to facilitate an efficient data exchange. The interface IF may be connected directly to the computing unit CU, which may be connected in turn to the memory unit MU. This arrangement may facilitate a flow of information from the interface IF to the memory unit MU via the computing unit CU.

The provision unit PRVS may be designed to process and store data. The interface IF may thereby act as the interface for input/output operations, the computing unit CU may perform calculation tasks, and the memory unit MU may store data or results.

FIG. 9 shows a schematic representation of an advantageous embodiment of a training unit TRS. The training unit TRS may also be designed to execute a proposed method for providing a trained function.

The training unit TRS may include a training interface TIF, a training computing unit TCU, and a training memory unit TMU. The training interface TIF may be designed to receive and/or transmit training examination data. The training computing unit TCU may be designed to perform calculations, particularly in order to apply and/or adjust the trained function. The training memory unit TMU may be designed to store data, particularly training examination data, parameters of the trained function, and/or interim results of the calculations.

The components of the training unit TRS may be connected to each other to facilitate an efficient data exchange. The training interface TIF may be connected to the training computing unit TCU, which may be connected in turn to the training memory unit TMU. This arrangement may facilitate a flow of data from the training interface TIF to the training memory unit TMU via the training computing unit TCU.

The training unit TRS may be designed to execute the acts of a proposed method for providing a trained function by the training interface TIF, the training computing unit TCU, and the training memory unit TMU being designed to execute the corresponding method acts.

Advantageously, the training interface TIF may be designed to capture training examination data TUD from a multitude of imaging-based training examinations of one or more training entities. Furthermore, the training computing unit TCU and/or the training memory unit TMU may be designed to apply the trained function TF to input data, which is based on training examination data TUD, wherein a respective abstract training vector TV is provided, and to adjust ADJ-TF at least one parameter of the trained function TF on the basis of the training examination data TUD and the abstract training vector TV by a supervised coding regression or a self-supervised adjustment of a coder-decoder structure.

Advantageously, the training unit TRS may facilitate efficient and structured implementation of the function training. The modular structure of the training unit TRS may allow flexible adjustment to different training scenarios and requirements.

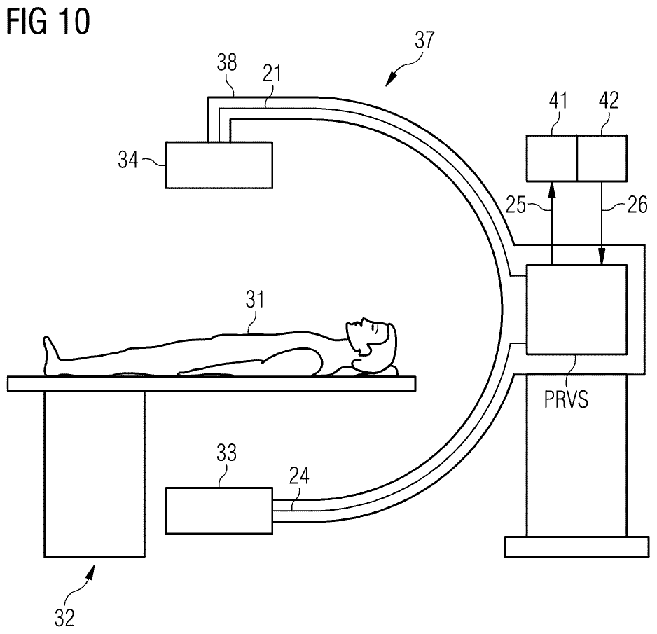

FIG. 10 shows a schematic representation of an advantageous embodiment of a system that includes a medical imaging device, particularly a C-arm X-ray device 37, and a provision unit PRVS.

The C-arm X-ray device 37 may be designed to record medical image data of the examination object 31, particularly to record X-ray projection recordings. An arm 38 of the C-arm X-ray device 37 may be positioned movably around one or more axes for recording the medical image data.

The system may also include a patient positioning device 32, on which the examination object 31 is arranged. The examination object 31 may be a human and/or animal patient and/or an examination phantom, for example, a vascular phantom.

The provision unit PRVS may send a signal 24 to an X-ray source 33 to record the medical image data. The X-ray source 33 may subsequently emit an X-ray beam. When an X-ray beam makes contact with a surface of a detector 34 after interacting with the examination object 31, the detector 34 may send a signal 21 to the provision unit PRVS. The provision unit PRVS may receive the medical image data using the signal 21. The examination data UD may include the medical image data.

Moreover, the C-arm X-ray device 37 may have an input unit 42, for example, a keyboard, and a display unit 41, for example, a monitor and/or display. The input unit 42 may be integrated into the display unit 41, for example, with a capacitive and/or resistive input display. The medical C-arm X-ray device 37 may thereby be controlled through an input of a medical operator, particularly of the first entity, on the input unit 42. Furthermore, other examination data UD of the imaging-based examination may be captured through an input of the medical operator on the input unit 42. The input unit 42 may send a signal 26 to the provision unit PRVS for this purpose. The display unit 41 may advantageously be designed to display a graphic representation of the examination data. The provision unit PRVS may send a signal 25 to the display unit 41 for this purpose.

The examination data UD may also include information about medical instruments that are used during the examination. This information may be captured and processed by the provision unit PRVS.

The provision unit PRVS may be designed to generate a profile of a doctor or a clinical specialty on the basis of identified image content. This profile may contain information about frequently performed examinations, medical instruments that have been used, or specific specialist knowledge. The examination data UD may advantageously include the profile.

The system may also include a mechanism that enables the first entity and/or the second entity, particularly the respective experts, to give feedback on how suitable the provided expertise of the second entity has been for the actual patient case. This feedback may be provided as user input via the input unit 42. The provision unit PRVS may collect the feedback and use it to improve the selection of experts. Additionally, the identification information ID may be output via the display unit 41.

The schematic representations in the described figures are not to scale and do not show proportions accurately.

Finally, it is noted once again that the method described in detail above and the illustrated devices merely concern exemplary embodiments, which may be modified by the person skilled in the art in a wide variety of ways without leaving the field of the disclosure. Moreover, use of the indefinite article “a” or “an” does not prevent the features concerned being present multiple times. Likewise, terms like “unit” and “element” do not exclude the possibility that the components concerned include multiple interacting subcomponents, which may be distributed, including spatially if applicable.

The expression “on the basis of” may be understood in the context of the present application in particular as “using.” In particular, stating that a first feature is produced (alternatively: established, provided, etc.) on the basis of a second feature does not exclude the possibility that the first feature may be produced (alternatively: established, provided, etc.) on the basis of a third feature.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A computer-implemented method for providing identification data for a second entity, the method comprising:
capturing examination data from an imaging-based examination of an examination object by a first entity;
providing an abstract vector by applying a trained function to input data, which is based on the examination data of the first entity, wherein the trained function is based on an abstract vector model; and
providing the identification data for the second entity based on a similarity comparison of the abstract vector with other abstract vectors in a common vector space stored in a collection, wherein respective identification data is assigned to the stored other abstract vectors.

2. The method of claim 1, wherein the examination data comprises:
medical image data of the examination object;
medical diagnostic data of the examination object;
medical anamnesis data of the examination object;
recording parameters of a medical imaging device for recording medical image data of the examination object;
operating parameters of a medical object during the imaging-based examination of the examination object;
measured values of a medical monitoring device;
a language of the medical diagnostic data and/or of the anamnesis data of the examination object;
metadata of the medical image data, and/or of the medical diagnostic data, and/or of the anamnesis data;
location information for the first entity; or
a combination thereof.

3. The method of claim 1, wherein some examination data less than all of the examination data coded in the abstract vector is excluded from the similarity comparison.

4. The method of claim 1, wherein the other abstract vectors with the respectively assigned identification data are stored in a data memory unit, a database, a cloud, or a combination thereof.

5. The method of claim 1, wherein the similarity comparison is based on a comparison of a geometric distance measurement between the respective abstract vectors in the common abstract vector space.

6. A computer-implemented method for providing a collection of abstract vectors, the method comprising:
capturing a multitude of examination data from imaging-based examinations of respective examination objects by respective reference entities;
applying a trained function to input data, which is based on the multitude of examination data, wherein a multitude of abstract vectors is provided in a common abstract vector space, and wherein the trained function is based on an abstract vector model;
collecting the multitude of abstract vectors in a data structure, wherein identification data for the respective reference entity is assigned to each of the abstract vectors in the data structure; and
providing the collection of abstract vectors.

7. The method of claim 6, wherein the examination data comprises:
medical image data of the respective examination object;
medical diagnostic data of the respective examination object;
medical anamnesis data of the respective examination object;
recording parameters of a medical imaging device;
operating parameters of a medical object during an imaging-based examination;
measured values of a medical monitoring device;
a language of the medical diagnostic data and/or of the anamnesis data of the respective examination object;
metadata of the medical image data, and/or of the medical diagnostic data, and/or of the anamnesis data;
location information for the reference entity; or
a combination thereof.

8. The method of claim 6, wherein the reference entities comprise medical facilities, competence centers, doctors, medical specialists, technical specialists, or a combination thereof

9. The method of claim 6, wherein the collecting of the multitude of abstract vectors comprises storage in a data memory unit, a database, a cloud, or a combination thereof.

10. The method of claim 6, further comprising:
updating the collection of abstract vectors by repeatedly adding new abstract vectors with respectively assigned identification data based on newly captured examination data.

11. The method of claim 10, wherein the updating is performed periodically or continually.

12. A computer-implemented method for providing a trained function, the method comprising:
providing training examination data for a multitude of imaging-based training examinations of one or more training entities;
applying the trained function to input data, wherein the input data is based on the training examination data, and wherein a respective abstract training vector is provided;
adjusting at least one parameter of the trained function based on the training examination data and the abstract training vector by a supervised coding regression or a self-supervised adjustment of a coder-decoder structure; and
providing the trained function.

13. The method of claim 12, wherein the training examination data comprises:
medical image data of a respective training examination object;
medical diagnostic data of the respective training examination object;
medical anamnesis data of the respective training examination object;
recording parameters of a medical imaging device;
operating parameters of a medical object during an imaging-based examination;
measured values of a medical monitoring device;
a language of the medical diagnostic data and/or of the anamnesis data of the respective training examination object;
metadata of the medical image data, and/or of the medical diagnostic data, and/or of the anamnesis data;
location information for the training entity; or
a combination thereof.

14. The method of claim 12, wherein the adjusting of the at least one parameter of the trained function comprises a federated adjustment of the at least one parameter of the trained function.

15. A system comprising:
a provision unit configured to:
receive examination data from an imaging-based examination of an examination object;
provide an abstract vector by applying a trained function to input data, which is based on the examination data, wherein the trained function is based on an abstract vector model; and
provide the identification data for an entity based on a similarity comparison of the abstract vector with other abstract vectors in a common vector space stored in a collection, wherein respective identification data is assigned to the stored other abstract vectors.

16. The system of claim 15, further comprising:
a medical imaging device configured to capture the examination data of the examination object and provide the examination data to the provision unit.
